(19) United States
(12) Patent Application Publication
(10) Pub. No.: US 2019/0177691 A1
(43) Pub. Date: Jun. 13, 2019

(54) MICROELECTRONIC HUMAN BLOOD BRAIN BARRIER

(71) Applicant: Stemonix Inc., Eden Prairie, MN (US)

(72) Inventor: Robert John Petcavich, The Woodlands, TX (US)

(21) Appl. No.: 16/321,908

(22) PCT Filed: Aug. 2, 2017
(86) PCT No.: PCT/US2017/045119

§ 371 Date: Jan. 30, 2019

Related U.S. Application Data
(60) Provisional application No. 62/370,101, filed on Aug. 2, 2016.

(51) Int. Cl. C12N 5/079; C12N 5/071; C12N 5/00; G01N 33/50

(52) U.S. Cl. CPC C12N 5/0622; C12N 5/069; C12N 5/0025; G01N 33/5058; C12N 2502/081; C12N 2529/00; C12N 2503/04; C12N 2513/00; C12N 2533/52; C12N 2533/12; C12N 2533/30; C12N 2502/28

(57) ABSTRACT

The present disclosure provides a planar microelectronic human blood brain barrier stack used to model drug effects and transport across the brain capillary endothelial barrier to neurons. In one embodiment the stack is comprised of a carrier substrate, electrode arrays, astrocytes, extracellular matrix and brain capillary endothelial cells.

### CROSS REFERENCE TO RELATED APPLICATIONS

This application claims the benefit of the filing date of U.S. application Ser. No. 62/370,101, filed on Aug. 2, 2017, the disclosure of which is incorporated by reference herein.

### FIELD OF THE DISCLOSURE

This disclosure relates to a method of creating a planar microelectronic multilayer cellular stack blood brain barrier (BBB) in vitro. The multilayer stack emulates a human blood-brain barrier (BBB) and can electronically measure neuron response to various therapeutic treatment challenges presented to the brain endothelial capillary cell side layer of the BBB stack.

### BACKGROUND

The blood-brain barrier (BBB) is a highly selective permeability barrier that separates the circulating blood from the brain extracellular fluid in the central nervous system (CNS). The blood-brain barrier is formed by brain endothelial cells, which are connected by tight junctions with an extremely high electrical resistivity of at least 0.1 Ω·m, and astrocytes. The blood-brain barrier allows the passage of water, some gases, and lipid-soluble molecules by passive diffusion, as well as the selective transport of molecules such as glucose and amino acids that allow for neural function. On the other hand, the blood-brain barrier may prevent the entry of lipophilic, potential neurotoxins by way of an active transport mechanism mediated by P-glycoprotein. A small number of regions in the brain, including the circumventricular organs (CVOs), do not have a blood-brain barrier.

The blood-brain barrier occurs along all capillaries and consists of tight junctions around the capillaries that do not exist in normal circulation. Endothelial cells restrict the diffusion of microscopic objects (e.g., bacteria) and large or hydrophilic molecules into the cerebrospinal fluid (CSF), while allowing the diffusion of small or hydrophobic molecules (O₂, CO₂, hormones and the like). Cells of the barrier actively transport metabolic products such as glucose across the barrier with specific proteins. This barrier also includes a thick basement membrane and astrocytic endfeet. This “barrier” results from the selectivity of the tight junctions between endothelial cells in CNS vessels that restricts the passage of solutes. At the interface between blood and the brain, endothelial cells are stitched together by these tight junctions, which are composed of smaller subunits, frequently biochemical dimers that are transmembrane proteins such as occluding, claudins, junctional adhesion molecule (JAM), or ESAM, for example. Each of these transmembrane proteins is anchored into the endothelial cells by another protein complex that includes zo-1 and associated proteins.

The blood-brain barrier is composed of high-density cells restricting passage of substances from the bloodstream much more than do the endothelial cells in capillaries elsewhere in the body. Astrocyte cell projections called astrocytic feet (also known as “glia limitans”) surround the endothelial cells of the BBB, providing biochemical support to those cells. The BBB is distinct from the quite similar blood-cerebrospinal fluid barrier that is a function of the choroidal cells of the choroid plexus, and from the blood-retinal barrier, which can be considered a part of the whole realm of such barriers.

Several areas of the human brain are not on the brain side of the BBB. Some examples of this include the circumventricular organs, the roof of the third and fourth ventricles, capillaries in the pineal gland on the roof of the diencephalon and the pineal gland. The pineal gland secretes the hormone melatonin “directly into the systemic circulation”, thus the blood-brain barrier, does not affect melatonin.

The blood-brain barrier acts very effectively to protect the brain from most pathogens. Thus, blood borne infections of the brain are very rare. Infections of the brain that do occur are often very serious and difficult to treat. Antibodies are too large to cross the blood-brain barrier, and only certain antibiotics are able to pass. In some cases, a drug has to be administered directly into the cerebrospinal fluid, (CSF), where it can enter the brain by crossing the blood-cerebrospinal fluid barrier. However, not all drugs that are delivered directly to the CSF can effectively penetrate the CSF barrier and enter the brain. The blood-brain barrier becomes more permeable during inflammation. This allows some antibiotics and phagocytes to move across the BBB. However, this also allows bacteria and viruses to infiltrate the BBB. Examples of pathogens that can traverse the BBB and the diseases they cause include Toxoplasma gondii which causes toxoplasmosis, spirochetes like Borrelia which causes Lyme disease, Group B streptococci which causes meningitis in newborns, and Treponema pallidum which causes syphilis. Some of these harmful bacteria gain access by releasing cytotoxins like pneumolysin which have a direct toxic effect on brain microvascular endothelium and tight junctions.

There are also some biochemical poisons that are made up of large molecules that are too big to pass through the blood-brain barrier. This was especially important in more primitive times when people often ate contaminated food. Neurotoxins such as botulinum in the food might affect peripheral nerves, but the blood-brain barrier can often prevent such toxins from reaching the central nervous system, where they could cause serious or fatal damage.

The blood-brain barrier (BBB) excludes from the brain about 100% of large-molecule neurotherapeutics and more than 98% of all small-molecule drugs. Overcoming the difficulty of delivering therapeutic agents to specific regions of the brain presents a major challenge to treatment of most brain disorders. In its neuroprotective role, the blood-brain barrier functions to hinder the delivery of many potentially important diagnostic and therapeutic agents to the brain. Therapeutic molecules and antibodies that might otherwise be effective in diagnosis and therapy do not cross the BBB in adequate amounts.

Mechanisms for drug targeting in the brain involve going either “through” or “behind” the BBB. Modalities for drug delivery/dosage form through the BBB entail its disruption by osmotic means; biochemically by the use of vasoactive substances such as bradykinin; or even by localized exposure to high-intensity focused ultrasound (HIFU). Other methods used to get through the BBB may entail the use of endogenous transport systems, including carrier-mediated transporters such as glucose and amino acid carriers; receptor-mediated transcytosis for insulin or transferrin; and the blocking of active efflux transporters such as p-glycoprotein. However, vectors targeting BBB transporters, such as the transferrin receptor, have been found to remain entrapped in brain endothelial cells of capillaries, instead of being ferried across the BBB into the cerebral parenchyma. Methods for drug delivery behind the BBB include intracerebral implantation (such as with needles) and convection-enhanced distribution. Mannitol can be used in bypassing the BBB.

### SUMMARY

The present disclosure provides for an in vitro BBB device having human cells that can measure the electrophysiology of neurons, in particular astrocytes, that are in physical contact or proximity with brain capillary endothelial cells. Exposing the device having stacks of such cells to therapeutic challenges and electronically measuring the effects of the therapeutic drug candidates to determine if they are transported across the BBB endothelial cells barrier to, in one embodiment, astrocytes. In one embodiment, therapeutic compounds such as small molecules and biomolecules for various brain related diseases are screened. In one embodiment, the device is not one where cells, e.g., different types of cells, are placed on two different sides of a non-biological material, such as a porous membrane. For instance the BBB device is not a transwell system.

In one embodiment, a viable and functional multilayer human cell stack is provided that includes an electrode, astrocytes; one or more of extracellular matrix or a component thereof, other biomolecules, or a synthetic polymer; and brain capillary endothelial cells.

In one embodiment, a multilayer human cell stack includes a multielectrode array; astrocytes; one or more of extracellular matrix, or a component thereof, other biomolecules, or a synthetic polymer; and brain capillary endothelial cells.

In one embodiment, a multilayer cell stack in combination with a multielectrode array to measure therapeutic drug effects such as impedance, action potentials and conduction velocities of neurons in contact with the conductive electrodes, is provided.

### BRIEF DESCRIPTION OF DRAWINGS

FIG. 1 shows a cross sectional view of one embodiment of the device.

### DETAILED DESCRIPTION

The following detailed description is directed towards the various embodiments of the invention. Although one or more of these embodiments may be preferred, the embodiments disclosed should not be interpreted, or otherwise used, as a limiting the scope of the disclosure, including the claims. In addition one skilled in the art will understand that the following description has broad application, and the discussion of any embodiment is meant only to be exemplary of that embodiment, and not intended to intimate that the scope of the disclosure, including the claims, is limited to that embodiment.

In one embodiment, multielectrode arrays (MEAs) in combination with multilayer cellular stacks of cells that are layered on top of the MEAs are used to measure electrophysiological changes in the neuron layer in contact with the MEA electrodes, as a result of potential therapeutic compounds crossing the capillary endothelial cells forming a barrier above the neuron cell layer.

In one embodiment, a microelectronic planar blood brain barrier device is provided. The device may include a planar substrate; one or more electrodes disposed on the planar substrate; a first layer comprising a plurality of mammalian neurons disposed on the one or more electrodes and also optionally the planar substrate; a second layer comprising one or more agents that are biocompatible and are disposed on at least some of the plurality of isolated neurons; and a third layer comprising a plurality of isolated endothelial cells disposed on the one or more agents. In one embodiment, the substrate, electrodes, or both, further include one or more cell binding molecules disposed thereon. In one embodiment, the cell binding molecules include a peptide or a polypeptide. In one embodiment, the substrate is formed of glass, silicon, standard printed circuit board (PCB), or flexible polymeric film. In one embodiment, the film is formed of Kapton, polycarbonate, or polyester (PET). In one embodiment, the thickness of the substrate is from about 1 micron to about 2 millimeters. In one embodiment, the thickness of the substrate is about 25 to 250 microns. In one embodiment, the thickness of the substrate is about 100 to 500 microns. In one embodiment, the one or more electrodes include copper, silver, gold, nickel, aluminum, indium tin oxide, graphene, carbon nanotubes, carbon nanobuds, or silver nanowires. In one embodiment, the electrodes have an electrical resistivity of less than 100 ohms per square. In one embodiment, the electrodes have an electrical resistivity of less than 50 ohms per square. In one embodiment, the electrodes have an electrical resistivity of less than 10 ohms per square. In one embodiment, the electrodes have an electrical resistivity of less than 5 ohms per square. In one embodiment, the mammalian neurons are astrocytes, e.g., human astrocytes. In one embodiment, the layer having the mammalian neurons is a single cell layer. In one embodiment, the layer having the mammalian neurons comprises 2 to 10 cell layers. In one embodiment, the one or more agents in the second layer include one or more of gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, or polyvinylprylidone. In one embodiment, the thickness of the second layer is from about 10 nanometers to 250 microns. In one embodiment, the thickness of the second layer is 0.5 to 5 microns. In one embodiment, the endothelial cells comprise capillary endothelial cells, e.g., human capillary endothelial cells. In one embodiment, the endothelial cells comprise brain capillary endothelial cells. In one embodiment, the layer having the mammalian endothelial cells is a single cell layer. In one embodiment, the layer having the mammalian endothelial cells comprises 2 to 10 cell layers. The device may be employed to screen compounds for their ability to cross the endothelial layer and alter the activity of the neurons in the device.

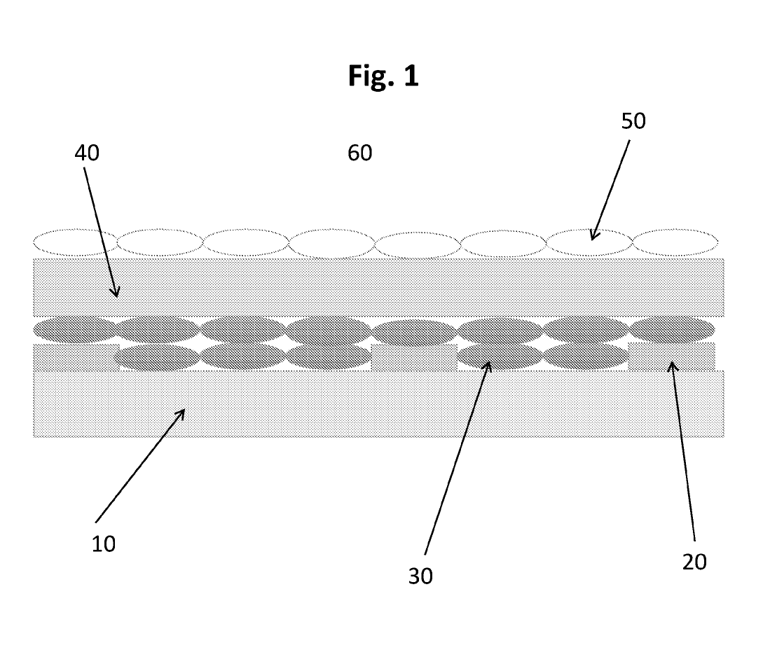

FIG. 1 shows a cross section of one embodiment. The multilayer stack 60 is comprised of an electrode support 10, conductive electrodes 20, neurons 30, extracellular matrix 40, and capillary endothelial cells 50.

The electrode support 10 can be formed of materials including but not limited to glass, silicon, standard printed circuit board (PCB), or flexible polymeric film such as Kapton, polycarbonate, or polyester (PET) film. The thickness of the support 10 may range from about 1 micron to about 2 millimeters, e.g., about 25 to 250 microns. The support 10 may be opaque or transparent and in one embodiment comprises transparent PET.

The conductive electrodes 20 may be formed of materials including but not limited to a conductor such as copper, silver, gold, nickel, aluminum, indium tin oxide, graphene, carbon nanotubes, carbon nanobuds, or silver nanowires. The electrodes 20 may have an electrical resistivity of less than 100 ohms per square, e.g., less than 10 ohms per square. The electrodes may be patterned in any geometric shape or size width lines, e.g., interdigitated conductive lines. The width of the lines may vary from about 1 to about 300 microns, e.g., about 50 to 100 microns. In one embodiment, copper electrodes 10 that have been flash plated with gold make the surface more biologically compatible for cell attachment and viability.

Once the multielectrode array 20 has been fabricated on a support material 10 the next step is to attach neurons to the electrodes, e.g., gold plated electrodes. Good cell adhesion and attachment allows for enhanced cell functioning, viability and measurement of the electrophysiology of the neurons during therapeutic drug exposures of the stack. In one embodiment, gold-coated copper electrodes 20 may be plasma cleaned to remove any surface contamination and then reacted with a 20 mM solution of alkanethiols of 11-mercaptoundecanoic acid (MUA) for 5 to 10 minutes. This results in a self assembled monolayer (SAM) or MUA on the surface. The electrodes may then be immersed into a 150 mM solution of 1-ethyl-3-(3-dimethylamino-propyl) carbodiimide (EDAC) and 30 mM N-hydroxysuccinimide (NHS) for 30 minutes to attach the NHS group to the terminus —COOH of the SAM layer. The finished activated electrode structure may then be sterilized with 70% ethanol for 15 minutes and exposed to various proteins that have binding sites for cells. For example, binding protein or polypeptides that may be used include but are not limited to fibronectin, laminin, Arg-Glu-Asp-Val-Tyr (REDV) or Lys-Arg-Glu-Asp-Val-Try (KREDVY). In one embodiment, KREDVY is employed to enhance cell binding and viability after cell attachment.

Neurons 30 are subsequently cultured by techniques well known in the art onto the protein-activated electrodes 20. There are many types of human neurons that can be used such as those derived from primary cells, or those derived from induced pluripotent stem cells (iPScs). There are about 10,000 specific types of neurons in the human brain but generally speaking they can be classified as motor neurons, sensory neurons, and interneurons. In one embodiment, astrocytes are employed as they play a role as the first layer of neurons adjacent to the brain capillary endothelial barrier (EB). Astrocytes process and modulate molecules that are transported through the EB before entering the brain. In one embodiment, iPSc derived astrocytes are employed as the neuron 30 layer.

Brain capillary endothelial cells (BCECs) 50 grow on extracellular matrix 40 in order to form very tight cell-to-cell contacts or junctions. A layer of extracellular matrix (ECM) 40 may be added between the neurons 30 and the BCECs. This is accomplished by applying a dilute solution 0.001 to 5% by weight in solution of the matrix into wells or chambers defined by the MEAs. Typical ECM components or synthetic polymers that can be used include but are not limited to gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylprylidone, or other polypeptides, or any combination of the aforementioned materials with or without crosslinking. The ECM layer may also contain adsorbed or absorbed polypeptides such as REDV and KREDVY to further enhance cell adhesion to the ECM or synthetic polymer containing layer. In one embodiment, gelatin and/or hyaluronic acid are the ECM components used in the ECM layer. The ECM may be deposited onto the astrocyte surface 30 and allowed to equilibrate for 12 to 24 hours before adding the last layer of the stack, the BCECs. The thickness of the ECM layer can range from 10 nanometers to 250 microns, e.g., 0.5 to 5 microns.

In one embodiment, the cells used for the BCEC layer are the hCMEC/D3 BBB cell line, which was derived from human temporal lobe microvessels and immortalized with hTERT and SV40 large T antigen. They are a model of human blood-brain barrier (BBB) function. The cell line is available from EMD Millipore Corporation in Temecula, Calif., is well characterized and easily cultured and grown. This BCEC layer 50 may be used to study pathological and drug transport mechanisms with relevance to the central nervous system.

Once the microelectronic planar BBB stack 60 is fabricated it may be used to study drug transport and effects on the astrocytes 30 that are bound to the MEA electrodes on the opposite planar surface to the BCEC layer. Electrophysiology properties of the astrocytes can be monitored and measured such as action potential, impedance, and conduction velocity. If drug or drug candidates are added to the BCEC side of the planar stack and if they pass through the BCEC layer their affect or lack thereof can be easily monitored electronically by the MEA array. Both drug efficacy and toxicity to both the BCEC and astrocyte layers may be measured.

In one embodiment, the in vitro BBB cell stack is in one or more wells of a plate, e.g., a multi-well plate, each having one or more electrodes on the bottom surface of the wells in contact with neurons in the cell stacks. The cell stack may be cultured in media or any physiologically compatible solution, or reside in a gel. One or more test compounds may be added to individual wells with cell stacks using, for example, micropipettes or an automated pipetting device.

In one embodiment, a substrate has a plurality of BBB cell stacks in a microarray having a plurality of electrodes, at least one of the electrodes in contact with neurons in the cell stacks. The substrate may be placed in a receptacle so that the cell stacks on the substrate may be cultured in media or any physiologically compatible solution.

The above discussion is meant to be illustrative of the principle and various embodiments of the present invention. Numerous variations and modifications will become apparent to those skilled in the art once the above disclosure id fully appreciated. It is intended that the following claims be interpreted to embrace all such variations and modifications.

1. A microelectronic planar blood brain barrier device, comprising:
a planar substrate;
one or more electrodes in contact with the planar substrate;
a first layer comprising a plurality of mammalian neurons in contact with the one or more electrodes and also optionally the planar substrate;
a second layer comprising one or more agents that are biocompatible and optionally adhere to at least some of the plurality of neurons; and
a third layer comprising a plurality of endothelial cells in contact with the one or more agents.

2. The device of claim 1 wherein the substrate further comprises one or more cell binding molecules.

3. The device of claim 2 wherein the molecules comprise a peptide or a polypeptide.

4. The device of claim 3 wherein the peptide or polypeptide includes fibronectin, laminin, Arg-Glu-Asp-Val-Tyr (REDV) or Lys-Arg-Glu-Asp-Val-Try (KREDVY).

5. The device of claim 1 wherein the substrate comprises glass, silicon, standard printed circuit board (PCB), or flexible polymeric film.

6. The device of claim 5 wherein the film comprises Kapton, polycarbonate, or polyester (PET).

7. The device of claim 1 wherein the thickness of the substrate is from about 1 micron to about 2 millimeters or about 25 to 250 microns.

8. (canceled)

9. The device of claim 1 wherein the one or more electrodes comprise copper, silver, gold, nickel, aluminum, indium tin oxide, graphene, carbon nanotubes, carbon nanobuds, or silver nanowires.

10. The device of claim 1 wherein the electrodes have an electrical resistivity of less than 100 ohms per square.

11. The device of claim 1 wherein the electrodes have an electrical resistivity of less than 10 ohms per square.

12. The device of claim 1 wherein the mammalian neurons are astrocytes.

13. The device of claim 12 wherein the astrocytes are human astrocytes.

14. The device of claim 1 wherein the one or more agents in the second layer include one or more of gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, or polyvinylprylidone.

15. The device of claim 1 wherein the thickness of the second layer is from about 10 nanometers to 250 microns or 0.5 to 5 microns.

16. (canceled)

17. The device of claim 1 wherein the endothelial cells comprise capillary endothelial cells.

18. The device of claim 17 wherein the endothelial cells comprise brain capillary endothelial cells.

19. (canceled)

20. The device of claim 1 wherein the one or more electrodes comprise gold plated copper and the one or more agents in the second layer include extracellular matrix.

21. The device of claim 2 wherein the one or more cell binding molecules comprise KREDVY.

22. A method of using a device, comprising:
providing the device of claim 1;
contacting the endothelial cells in the device with one or more test compounds; and
detecting whether the one or more compounds alter the activity of the neurons in the device.

23. The method of claim 22 wherein the activity detected is action potential, impedance or conduction velocity.
